(19) United States
(12) Patent Application Publication
(10) Pub. No.: US 2026/0004480 A1
(43) Pub. Date: Jan. 1, 2026

(54) CT APPARATUS, OPERATION METHOD OF CT APPARATUS, AND OPERATION PROGRAM OF CT APPARATUS

(71) Applicant: FUJIFILM Corporation, Tokyo (JP)

(72) Inventors: Kana KOBAYASHI, Tokyo (JP); Taiga Goto, Tokyo (JP)

(73) Assignee: FUJIFILM Corporation, Tokyo (JP)

(21) Appl. No.: 19/242,922

(22) Filed: Jun. 18, 2025

(30) Foreign Application Priority Data
Jun. 28, 2024 (JP) 2024-105410

(51) Int. Cl. G06T 11/00; A61B 6/00; A61B 6/03; A61B 6/46; G06T 7/246

(52) U.S. Cl. CPC G06T 11/003; A61B 6/032; A61B 6/461; A61B 6/544; G06T 7/246; G06T 2207/10081; G06T 2207/30004; G06T 2210/41

(57) ABSTRACT

A CT apparatus sets a subject region in an imaging range of a radiation image by using information related to a subject, calculates, before main imaging of the subject, a size of a peripheral region, around the subject region and in a range outside the subject region, in which the subject is estimated to protrude from the subject region due to body movement, as a margin degree with respect to the subject region, and outputs, during the main imaging of the subject, projection data of a channel corresponding to a range of an enlarged subject region, which is obtained by adding the peripheral region to the subject region, to a console.

### CROSS-REFERENCE TO RELATED APPLICATIONS

This application claims priority under 35 USC 119 from Japanese Patent Application No. 2024-105410 filed on Jun. 28, 2024, the disclosure of which is incorporated by reference herein.

### BACKGROUND

### 1. Technical Field

The technology of the present disclosure relates to a CT apparatus, an operation method of a CT apparatus, and an operation program of a CT apparatus.

### 2. Related Art

JP2013-22427A discloses an X-ray diagnostic apparatus that calculates a subject data ratio indicating a ratio of a data region in which X-rays are transmitted through a subject for a plurality of slice rows of front scan image data and side scan image data of the subject captured by an X-ray detector, that calculates a view rate based on the subject data ratio, that determines a data region in which the X-rays are not transmitted through the subject from projection data of the subject captured based on the view rate, and that encodes the projection data in which the data region is reduced into transmission data.

JP2022-26909A discloses a medical image diagnostic apparatus that collects detection data having a first resolution related to a subject and that outputs first detection data corresponding to a first region of the subject and having the first resolution and second detection data corresponding to a second region of the subject and having a second resolution lower than the first resolution, in the collected detection data, in association with detection region information.

### SUMMARY

For example, in a photon counting CT apparatus, as the number of photoelectric conversion elements in a radiation detector increases, there is a tendency that an output time for outputting projection data from the radiation detector to a console increases.

Therefore, instead of outputting the entire projection data to the console, only the projection data corresponding to a subject region and a peripheral region of a predetermined range extending outward from the subject region in the projection data is output to the console, and thus an output data amount may be reduced.

Meanwhile, a range of the subject region is set based on a scanogram image obtained by imaging the subject in advance before main imaging in order to determine an imaging range of the subject by the CT, but a position of the subject during the main imaging may change from a position at the time of capturing the scanogram image. Therefore, the projection data of the peripheral region of the subject region is also output to the console in addition to the subject region, but the magnitude of the body movement varies depending on a part of the subject.

Therefore, in a case in which a range of the peripheral region is fixed to the predetermined range, a situation may occur in which a part of the subject is not included in the projection data output to the console.

The technology according to the present disclosure provides a CT apparatus, an operation method of a CT apparatus, and an operation program of a CT apparatus capable of outputting projection data including an entire subject even in a case in which an output data amount of the projection data to be output to a console is reduced.

The technology of the present disclosure relates to a CT apparatus comprising: a processor, in which the processor is configured to: set a subject region in an imaging range of a radiation image by using information related to a subject; calculate, before main imaging of the subject, a size of a peripheral region, around the subject region and in a range outside the subject region, in which the subject is estimated to protrude from the subject region due to body movement, as a margin degree with respect to the subject region; and output, during the main imaging of the subject, projection data of a channel corresponding to a range of an enlarged subject region, which is obtained by adding the peripheral region represented by the margin degree to the subject region, to a console.

It is preferable that the processor is configured to calculate the margin degree in accordance with a part of the subject to be imaged.

It is preferable that the processor is configured to calculate the margin degree in accordance with an attribute of a person who is the subject.

It is preferable that the processor is configured to estimate the attribute of the subject from an optical image obtained by imaging the subject.

It is preferable that the processor is configured to reset a display range of the subject region set in advance, in accordance with an instruction of an operator, before the main imaging of the subject.

It is preferable that the processor is configured to set the subject region based on a tomographic model of the subject generated using pre-generation data used for generating a scanogram image or an optical image obtained by imaging the subject.

It is preferable that the processor is configured to calculate the margin degree for each imaging direction of the subject, and output, during the main imaging of the subject, projection data of a channel corresponding to a range of the subject region to which the peripheral region represented by the margin degree is added, to the console.

It is preferable that the processor is configured to output projection data including a specific channel to be referred to by the console to reconstruct a tomographic image of the subject, in addition to the channel corresponding to the range of the enlarged subject region, to the console.

It is preferable that the processor is configured to control an irradiation field limiter that limits a radiation irradiation field such that an outer side of the range of the enlarged subject region is not irradiated with radiation.

It is preferable that the processor is configured to adjust a display range of a tomographic image of the subject, which is reconstructed from the projection data, in accordance with the number of channels included in the projection data received by the console, and display the tomographic image of the subject of which the display range is adjusted, on a display device.

It is preferable that the processor is configured to readjust the display range adjusted in accordance with the number of channels included in the projection data, in accordance with an instruction from an operator.

It is preferable that the processor is configured to set a pixel value of a pixel in a range corresponding to a region outside the enlarged subject region in the tomographic image of the subject, to a predetermined value.

The technology of the present disclosure relates to an operation program of a CT apparatus, the operation program causing a computer to execute a process comprising: setting a subject region in an imaging range of a radiation image by using information related to a subject; calculating, before main imaging of the subject, a size of a peripheral region, around the subject region and in a range outside the subject region, in which the subject is estimated to protrude from the subject region due to body movement, as a margin degree with respect to the subject region; and outputting, during the main imaging of the subject, projection data of a channel corresponding to a range of an enlarged subject region, which is obtained by adding the peripheral region represented by the margin degree to the subject region, to a console.

The technology of the present disclosure relates to an operation method of a CT apparatus, the operation method being executed by a computer, the operation method comprising: setting a subject region in an imaging range of a radiation image by using information related to a subject; calculating, before main imaging of the subject, a size of a peripheral region, around the subject region and in a range outside the subject region, in which the subject is estimated to protrude from the subject region due to body movement, as a margin degree with respect to the subject region; and outputting, during the main imaging of the subject, projection data of a channel corresponding to a range of an enlarged subject region, which is obtained by adding the peripheral region represented by the margin degree to the subject region, to a console.

According to the technology of the present disclosure, it is possible to output the projection data including the entire subject even in a case in which the output data amount of the projection data to be output to the console is reduced.

### BRIEF DESCRIPTION OF THE DRAWINGS

Exemplary embodiments of the technology of the disclosure will be described in detail based on the following figures, wherein:

FIG. 1 is a diagram showing an apparatus configuration example of a CT apparatus;

FIG. 2 is a diagram showing a functional configuration example of the CT apparatus;

FIG. 3 is a diagram showing an example of a flowchart related to output processing of projection data to a console;

FIG. 4 is a diagram showing examples of a subject region, a peripheral region, and an enlarged subject region;

FIG. 5 is a diagram showing an example of projection data including a reference channel; and

FIG. 6 is a diagram showing an example of a flowchart of image display processing.

### DETAILED DESCRIPTION

Hereinafter, the present embodiment of the present invention will be described in detail with reference to the drawings. It should be noted that the same components and the same processing are denoted by the same reference numerals throughout all the drawings, and the redundant description will be omitted. Dimensional ratios in the drawings are exaggerated for convenience of description and may be different from the actual ratios.

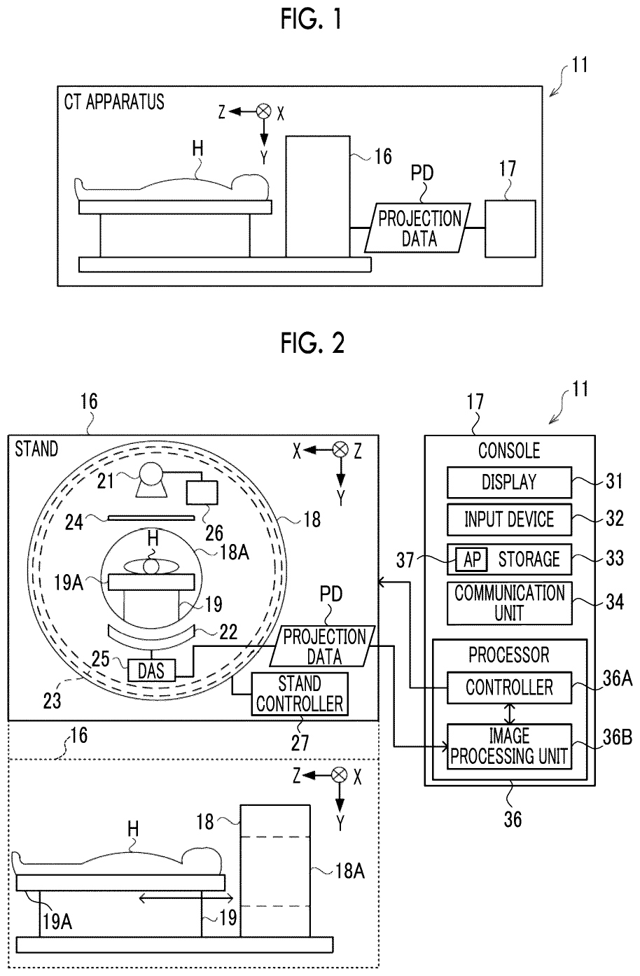

FIG. 1 is a diagram showing an apparatus configuration example of a CT apparatus 11 according to the technology of the present disclosure. The CT apparatus 11 obtains a tomographic image of a subject by imaging a part of a person under examination H to be imaged, that is, a subject using radiation (for example, X-rays). The CT apparatus 11 is installed in an imaging room of a radiology department in a medical facility as an example. The CT apparatus 11 comprises a stand 16 and a console 17. The console 17 functions as an operation terminal and a control device for operating the stand 16. The console 17 is operated by an operator such as a radiological technologist. Further, the console 17 also functions as an image processing device that performs image processing on data output from the stand 16 to generate the tomographic image. Further, the console 17 also functions as a display device that displays the generated tomographic image.

FIG. 2 is a diagram showing a functional configuration example of the CT apparatus 11. As shown in FIG. 2, the stand 16 is a main portion of the CT apparatus 11, and comprises a gantry 18 and an examination table device 19. In FIG. 2, in addition to a diagram in a front view of the stand 16, a diagram in a side view of the stand 16 is shown in a rectangular broken line frame. The examination table device 19 includes a top plate 19A on which the person under examination H can be placed in a decubitus posture. The person under examination H is placed in a posture in which a body axis matches a longitudinal direction of the top plate 19A (Z axis direction of the stand 16). The top plate 19A can be moved in the Z axis direction. The gantry 18 has an annular shape as a whole, and a circular opening portion 18A having a diameter larger than a width of the top plate 19A is formed at the center. During the imaging, the top plate 19A on which the person under examination His placed is moved in the Z axis direction with respect to the gantry 18 to enter the opening portion 18A. The imaging is performed while moving the top plate 19A with respect to the gantry 18.

A radiation source 21, a detector 22, and a frame 23 are disposed inside the gantry 18. The radiation source 21 emits the radiation toward the person under examination H. The detector 22 is a radiation detector that detects the radiation transmitted through the person under examination H. The radiation transmitted through the person under examination His attenuated by interaction (absorption, scattering, and the like of the radiation) with structures such as organs and bones in the body of the person under examination H. The structures each have an attenuation coefficient for the radiation peculiar to the structures, and the radiation transmitted through the structure carries intensity information reflecting the attenuation coefficient of the structure. The detector 22 has a detection surface in which pixels are two-dimensionally arranged, and outputs a detection signal in accordance with the intensity information of the radiation for each pixel in the detection surface. Further, the detector 22 has a substantially arc shape in accordance with a curvature of the gantry 18, and the detection surface is also curved.

The radiation source 21 and the detector 22 are disposed at positions facing each other in the gantry 18 and are rotated about the Z axis while maintaining a facing posture. The frame 23 has an annular shape and supports the radiation source 21 and the detector 22 in a rotatable manner. During the imaging, the stand 16 detects projection data PD at a plurality of positions in a circumferential direction about the Z axis corresponding to the body axis of the person under examination H while rotating the radiation source 21 and the detector 22 about the person under examination H on the top plate 19A, by using the detector 22. During the imaging, the top plate 19A is also moved in the Z axis direction in synchronization with the rotation of the radiation source 21 and the detector 22. As a result, the projection data PD of the radiation at each position about the body axis of the person under examination H is acquired.

In the stand 16, a Y axis represents a height direction, and an X axis represents a width direction. As an example, in the examination table device 19, the person under examination H is placed in a decubitus posture with a head side facing the gantry 18. Therefore, in the imaging, the person under examination H enters the gantry 18 from the head side, and the projection data PD is output in the order from the head side to a foot side.

A data acquisition system (DAS) 25 collects the detection signal output by the detector 22, generates the projection data PD at each position about the Z axis based on the collected detection signal, and outputs the generated projection data PD to the console 17.

The detector 22 is a multi-slice type radiation detector. The multi-slice type radiation detector is a radiation detector in which a plurality of photoelectric conversion elements are arranged along the body axis direction of the person under examination H, that is, a slice direction, and a direction orthogonal to the slice direction, that is, a channel direction. As an example, m photoelectric conversion elements are arranged in the slice direction and n photoelectric conversion elements are arranged in the channel direction to form a matrix of m rows×n columns. Therefore, the DAS 25 outputs the projection data PD of the maximum m rows×n columns to the console 17.

An irradiation field limiter 24 (also referred to as a collimator) that limits a radiation irradiation field is disposed in front of the radiation source 21 in an irradiation direction. The irradiation field limiter 24 has an irradiation opening of which a contour is defined by a plurality of shielding plates that shield the radiation, and a size of a irradiation opening can be changed by moving the shielding plates. A high-voltage generator 26 generates a high voltage to be supplied to the radiation source 21. The radiation source 21 and the detector 22 are electrically connected to the frame 23 by a slip ring method, and, for example, power supply, transmission and reception of data, and the like are performed via a slip ring. The imaging can be performed while rotating the radiation source 21 and the detector 22 in one direction without reversing the rotation direction of the radiation source 21 and the detector 22 by the connection using the slip ring method.

The stand 16 is provided with a stand controller 27. The stand controller 27 controls each portion of the stand 16 in addition to the rotation of the radiation source 21 and the detector 22 and the movement of the top plate 19A, based on an instruction from the console 17.

The imaging conditions of the CT apparatus 11 are set through the stand controller 27 via the operation from the console 17. The imaging conditions include an imaging range, a slice interval, and the like in addition to a radiation irradiation condition of the radiation source 21. The radiation irradiation condition includes a tube voltage (unit: kv) applied to the radiation source 21, a tube current (unit: mA), and a radiation irradiation time (unit: msec). The product of the tube current and the irradiation time defines a total radiation irradiation amount and is called a mAs value. The irradiation field is adjusted by, for example, changing the size of the irradiation opening of the irradiation field limiter 24 in an X-Z plane. In a case in which the radiation emitted from the radiation source 21 is a conical cone beam, a width of the irradiation field in the Z axis direction can be adjusted by adjusting a width of the irradiation opening of the irradiation field limiter 24 in the Z axis direction. In addition, the imaging range of the person under examination H in the Z axis direction, such as an entire body or a chest to an abdomen, is adjusted by changing a movement range of the top plate 19A.

Further, it is also possible to change an imaging range of a slice image. The slice image is a tomographic image representing an axial cross section orthogonal to an X-Y plane, that is, the body axis of the person under examination H, and is generated by image reconstruction based on the projection data PD as described later. The imaging range of such a slice image in the X-Y plane is also referred to as a field of view (FOV). For example, in the axial cross section of the chest of the person under examination H, a size of the FOV can be changed such that a relatively large region in which the entire chest is included is set as the FOV or such that a relatively small region such as a part of the chest is set as the FOV.

Meanwhile, since the number of pixels of the detector 22 is determined, a resolution of the slice image to be captured is increased in a case in which the FOV is reduced, and the resolution is decreased in a case in which the FOV is increased. For example, in a case in which the total number of pixels of the detector 22 that can be output to the console 17 (in this case, m×n pixels) is determined to be a predetermined value, the number of pixels n in the channel direction has to be reduced in a case in which the number of pixels m in the slice direction is increased. In a case in which the FOV can be reduced, the channel interval is made finer while the number of pixels n in the channel direction is maintained, and a resolution in a slice plane is increased. Alternatively, the number of pixels in the slice direction is increased instead of reducing the number of pixels in the channel direction, so that the slice interval can be made finer, and the resolution in the body axis direction can be increased.

The console 17 comprises a display 31, an input device 32, a storage 33, a communication unit 34, and a processor 36. The console 17 is configured based on, for example, a personal computer, and has a hardware configuration that is the same as a hardware configuration of a general computer. The display 31 is, for example, a liquid crystal display, and displays an operation screen, a captured slice image, and the like. The input device 32 is a device for the operator to input an operation instruction and is configured by a keyboard, a mouse, and the like.

The storage 33 is a data storage that stores various programs such as a control program for controlling each unit of the console 17. The various programs include an application program (AP) 37 causing the processor 36 to function as a control device and an image processing device of the CT apparatus 11. Examples of the storage 33 include a hard disk drive (HDD) and a solid-state drive (SSD). The projection data PD acquired from the stand 16 and the generated slice image are also temporarily stored in the storage 33. The application program 37 is an example of an “operation program” according to the technology of the present disclosure.

The communication unit 34 is a communication interface for performing communication between the console 17 and an external device, such as an image database (DB) outside the stand 16. The communication unit 34 is connected to a network (not shown), such as a local area network (LAN) and/or a wide area network (WAN), and performs transmission control in accordance with a communication protocol defined in various wired or wireless communication standards.

The processor 36 functions as a controller 36A that controls each unit of the console 17 and an image processing unit 36B that executes various types of image processing. The processor 36 is configured by, as an example, a central processing unit (CPU) and a memory, such as a random access memory (RAM). The CPU functions as the processor 36 by loading the various programs including the application program 37 from the storage 33 into the memory, and executing the loaded programs. The processor 36 is an example of a “processor” according to the technology of the present disclosure.

The controller 36A controls the stand 16 through the stand controller 27 in accordance with the instruction of the operator input from the input device 32. The image processing unit 36B executes slice image generation processing and multi-planar reconstruction (MPR) image generation.

The slice image generation processing is processing of generating the tomographic image by performing image reconstruction based on the projection data PD acquired from the stand 16. The reconstructed tomographic image is a tomographic image representing an axial cross section perpendicular to the body axis (Z axis) of the person under examination H.

An MPR image is a tomographic image representing a cross section of the person under examination H that is arbitrarily designated. The MPR image includes images of a sagittal cross section that is a vertical cross section of the person under examination H and a coronal cross section that is a horizontal cross section of the person under examination H, in addition to the axial cross section. The MPR image generation processing is processing of designating any cross section with respect to an isotropic three-dimensional image and cutting out the designated any cross section to generate the MPR image. The isotropic three-dimensional image is an image obtained by performing isotropic processing of making resolutions in the X axis direction, the Y axis direction, and the Z axis direction the same based on a slice volume including all the slice images in the imaging range.

Hereinafter, the slice image and the MPR image will be collectively referred to as a “tomographic image”. The tomographic image is an example of a “radiation image” according to the technology of the present disclosure, and is also an example of a “tomographic model”.

Next, output processing of the projection data PD to the console 17 in the CT apparatus 11 will be described.

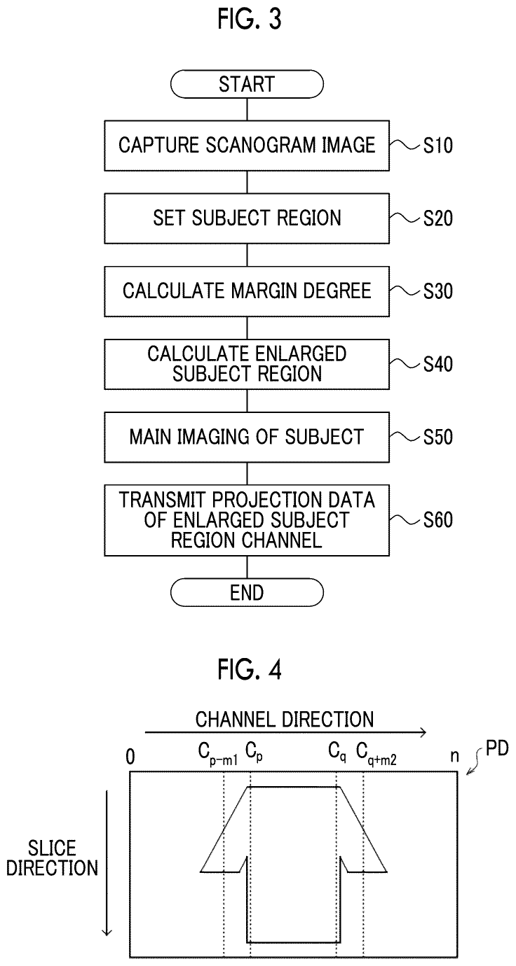

FIG. 3 is a diagram showing an example of a flowchart related to the output processing of the projection data PD to the console 17 executed by the CT apparatus 11 in a case in which the operator issues an imaging instruction for the subject.

It should be noted that the operator inputs information related to the subject through the input device 32 before giving the imaging instruction. The information related to the subject includes, for example, an attribute of the person under examination H and information related to an imaging part of the person under examination H for the tomographic image.

The attribute of the person under examination H is information indicating what kind of person the person under examination H is. The attribute of the person under examination H includes, for example, symptoms of the person under examination H, such as the age, sex, and presence or absence of dementia of the person under examination H.

In addition, the information related to the imaging part of the person under examination H for the tomographic image includes, for example, a name of the part of the person under examination H for which the tomographic image is captured, the presence or absence of a tumor, and the like.

In step S10, the processor 36 captures a scanogram image of the person under examination H placed on the top plate 19A. The scanogram image is an image obtained by imaging the person under examination H in advance in order to determine the imaging range of the radiation image before main imaging of the person under examination H by the CT apparatus 11.

In step S20, the processor 36 extracts the contour of the subject from the scanogram image or the axial cross section captured by the processing of step S10 to set a subject region.

It should be noted that the processor 36 displays the FOV for the main imaging on the display 31 by superimposing the FOV for the main imaging on the scanogram image or the axial cross section. The operator can further adjust the FOV displayed on the display 31 before the main imaging. In this case, the processor 36 resets the adjusted FOV, which is adjusted by the operator, as the subject region.

The processor 36 may extract the contour of the subject from scanogram raw data, instead of the scanogram image or the axial cross section, to set the subject region. The scanogram raw data is data including the projection data PD of all channels C generated in the DAS 25, and is an example of “pre-generation data used for generating a scanogram image” according to the technology of the present disclosure.

In a case of generating the scanogram image, the range to be imaged varies based on the FOV designated for the scanogram image, that is, a scanogram FOV, and thus the projection data PD of all the channels corresponding to the subject region is not always included in the scanogram image. Therefore, the setting accuracy of the subject region may be higher in a case in which the subject region is set from the scanogram raw data than in a case in which the subject region is set from the scanogram image or the axial cross section.

In addition, the processor 36 may extract the contour of the subject from the optical image of the person under examination H captured along the body axis by, for example, a visible light camera to set the subject region.

It should be noted that, in a case in which the person under examination H does not move until the main imaging, the position of the subject does not change. Therefore, in a case in which the projection data PD in a range corresponding to the subject region set by the processing of step S20 is extracted from the projection data PD obtained by the main imaging, and is output to the console 17, the tomographic image of the subject is obtained. However, in a case in which the person under examination H moves after the imaging of the scanogram image, and the range of the projection data PD to be output to the console 17 is determined in accordance with the set range of the subject region, a situation may occur in which the entire subject is not included in the tomographic image.

Therefore, in step S30, the processor 36 calculates a margin degree with respect to the subject region. The margin degree with respect to the subject region is a size of a peripheral region, around the subject region and in a range outside the subject region, in which the subject is estimated to protrude from the subject region due to body movement.

For example, the processor 36 calculates the margin degree in accordance with the part of the subject to be imaged. In a case in which the subject is the chest, the body movement is caused by the breathing of the person under examination H, but the body movement is small as compared with a part such as the head. Therefore, the processor 36 calculates the margin degree for each part in accordance with the part of the subject. Specifically, in a case in which the subject is the chest, 5% of the number of channels corresponding to the subject region is set as the margin degree. The number of channels corresponding to the subject region is the number of channels included in a range from one end portion to the other end portion of the subject region in the channel direction in the projection data PD. On the other hand, in a case in which the subject is the head, 10% of the number of channels corresponding to the subject region is set as the margin degree.

The margin degree associated with each part is stored in the storage 33 in advance, and the processor 36 may acquire the margin degree corresponding to the part to be imaged, which is input by the operator as the information related to the subject from the storage 33. The margin degree associated with each part stored in the storage 33 can be corrected by the operator. Therefore, the margin degrees of 5% and 10% shown above are merely examples, and there is no restriction on the value of the margin for each part.

It should be noted that the processor 36 may set the margin degree with respect to the subject region from elements other than the part of the subject. For example, children tend to move the body more than adults. Therefore, in a case in which the age of the person under examination H is included in a range set in advance as the age of the child, it is preferable that the processor 36 sets a value larger than the margin degree set for the person under examination H, who is an adult, as the margin degree with respect to the subject region even in a case in which the subject is the same part. For example, in a case in which the age of the person under examination H is 0 years old or older and younger than 12 years old, the processor 36 determines that the person under examination H is the child. The range of the age of the child is stored in the storage 33 in advance, and can be modified by the operator.

In addition, for example, in a case in which the person under examination H is recognized as having dementia, even in a case in which the operator gives an instruction not to move the body, the person under examination H may not be able to understand the instruction content, so that the body movement tends to be larger than that of the person under examination H who is not recognized as having dementia. Therefore, in a case in which the person under examination H is recognized as having dementia, it is preferable that the processor 36 sets a value larger than the margin degree set for the person under examination H who is not recognized as having dementia, as the margin degree with respect to the subject region.

As described above, the processor 36 may set the margin degree with respect to the subject region in accordance with the attribute of the person under examination H, such as the age or the symptom. It goes without saying that the processor 36 may set the margin degree with respect to the subject region by combining the part of the subject and the attribute of the person under examination H.

The attribute of the person under examination H, such as the age or the symptom, may be acquired from the information related to the subject input by the operator, but the processor 36 may estimate the attribute of the person under examination H that can be identified from the appearance by using the optical image obtained by imaging the person under examination H. For capturing the optical image, for example, a visible light camera (not shown), which captures visible light and images the visible light, is used. The processor 36 estimates the attribute of the person under examination H that is identifiable from the appearance, such as the age of the person under examination H, from the optical image of the person under examination H captured by the visible light camera using a known image recognition method.

Further, the processor 36 may set the margin degree with respect to the subject region from the imaging condition of the tomographic image set by the operator, that is, a CT imaging protocol.

In step S40, the processor 36 calculates an enlarged subject region obtained by adding the peripheral region represented by the margin degree set by the processing of step S30 to the subject region. The processor 36 stores the calculated enlarged subject region in the memory.

The processor 36 calculates a channel C corresponding to a range of the enlarged subject region based on the enlarged subject region. The channel C corresponding to the range of the enlarged subject region is the channel C that constitutes a minimum range including all the peripheral regions and the subject region in the projection data PD.

FIG. 4 is a diagram showing examples of the subject region, the peripheral region, and the enlarged subject region in the projection data PD in a case in which the person under examination H is scanned along the Z axis. In FIG. 4, a range from a channel Cₚ to a channel C_{q} in the channel direction is defined as the subject region. It should be noted p and q (0<p<q) are integers representing channel numbers for identifying the channel C, and take values of 0 or more and n (q<n) or less. Here, m1 and m2 are integers of 1 or more.

In this case, a range of the channel C that is Cₚ₋ₘ₁ or more and less than Cₚ and a range of the channel C that is more than C_{q} and C_{q+m2} or less are the peripheral region. Further, a range of the channel C of Cₚ₋ₘ₁ or more and C_{q+m2} or less, which is the sum of the subject region and the peripheral region, is the enlarged subject region. That is, in the example of FIG. 4, the channel C corresponding to the range of the enlarged subject region is a set of the channels C composed of photoelectric conversion element columns corresponding to each channel C of Cₚ₋ₘ₁ or more and C_{q+m2} or less among n photoelectric conversion elements arranged in the channel direction.

In a case in which an instruction for the main imaging is received from the operator, in step S50 of FIG. 3, the processor 36 performs the main imaging of the subject in accordance with the CT imaging protocol set by the operator. As a result, the projection data PD of m rows×n columns is generated in the DAS 25.

In step S60, the processor 36 acquires the enlarged subject region stored in the memory by the processing of step S40 and outputs only the projection data PD composed of the channels C corresponding to the range of the enlarged subject region among the m rows×n columns of the projection data PD to the console 17. Information indicating the range of the enlarged subject region is added to each of the projection data PD output to the console 17.

As described above, the output processing of the projection data PD to the console 17 shown in FIG. 3 ends.

### Modification Example 1 of Output Processing of Projection Data PD

In the processing of step S30 shown in FIG. 3, the processor 36 may calculate the margin degree for each imaging direction of the subject, that is, for each rotation direction of the radiation source 21 or for each circumferential direction about the body axis of the person under examination H. For example, in a case in which the subject is the chest, the body movement caused by breathing is larger in the Y axis direction than in the X axis direction. Therefore, the processor 36 may set the margin degree in a case in which the person under examination H is imaged along the X axis to 5%, and set the margin degree in a case in which the person under examination H is imaged along the Y axis to 7%.

By calculating the margin degree for each imaging direction of the subject, a larger margin degree can be set for a direction in which the body movement is likely to occur than in other directions. Therefore, it is possible to increase a probability that the entirety of the subject is included in the projection data PD output to the console 17, as compared with a case in which the margin degree is set without considering the imaging direction of the subject.

### Modification Example 2 of Output Processing of Projection Data PD

In a case in which the tomographic image is reconstructed in the console 17 based on the projection data PD, the reconstruction may be performed with reference to a specific channel C. For convenience of description, the channel C referred to as a reference will be particularly referred to as a “reference channel Cₓ”.

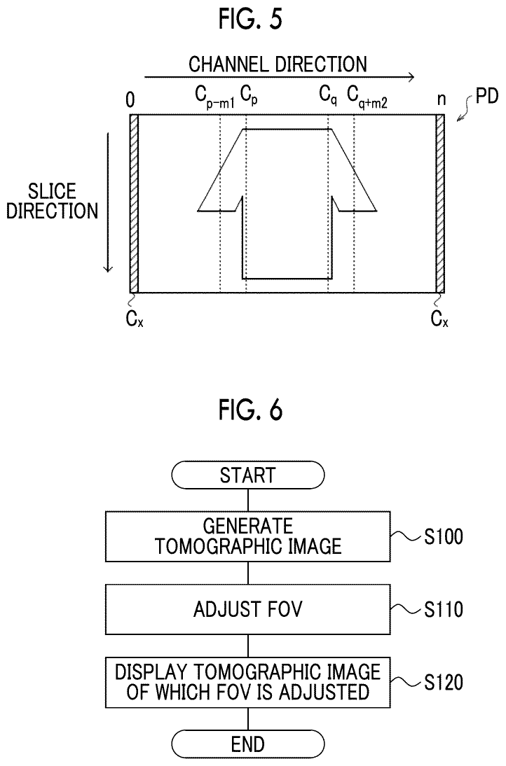

FIG. 5 is a diagram showing an example of the projection data PD including the reference channel Cₓ. The reference channel Cₓ shown in FIG. 5 represents an end portion of the projection data PD. For example, in a case in which the console 17 corrects the projection data PD using the reference channel Cₓ, it is necessary to include the reference channel Cₓ in the projection data PD. Therefore, the processor 36 includes, in the projection data PD, the reference channel Cₓ that is referred to by the console 17 to reconstruct the tomographic image of the subject, in addition to the channel C corresponding to the range of the enlarged subject region, and outputs the projection data PD to the console 17.

It should be noted that the position of the reference channel Cₓ shown in FIG. 5 is an example, and the reference channel Cₓ is not limited to the channel C corresponding to the end portion of the projection data PD.

### Modification Example 3 of Output Processing of Projection Data PD

In order to reduce an amount of exposure of the person under examination H caused by the capture of the tomographic image, it is preferable to avoid irradiating a part other than the subject with radiation as much as possible.

Therefore, in a case in which the main imaging of the subject is performed in the processing of step S50 in FIG. 3, the processor 36 may control the irradiation field limiter 24 such that an outer side of the range of the enlarged subject region is not irradiated with the radiation based on the enlarged subject region calculated in the processing of step S40 in FIG. 3.

### Reconstruction Processing of Tomographic Image

FIG. 6 is a diagram showing an example of a flowchart of image display processing executed by the console 17 that receives the projection data PD.

In step S100, the processor 36 performs the image reconstruction based on the acquired projection data PD to generate the tomographic image of the subject.

In step S110, the processor 36 adjusts the FOV of the tomographic image of the subject reconstructed from the projection data PD in accordance with the number of channels included in the projection data PD. As a result, a display range of the tomographic image is autonomously adjusted by the processor 36, and the tomographic image in which the entire subject is displayed as large as possible is obtained.

In step S120, the processor 36 displays the tomographic image of the subject of which the FOV is adjusted, on the display 31.

As described above, the image display processing shown in FIG. 6 ends.

It should be noted that the operator can also readjust the FOV of the tomographic image displayed on the display 31, as necessary. In this case, the processor 36 displays the tomographic image in accordance with the adjusted FOV, which is adjusted by the operator, on the display 31.

In addition, in a case in which the tomographic image of the subject is displayed on the display 31, the processor 36 may set a pixel value of a pixel in a range corresponding to a region outside the enlarged subject region to a predetermined value (for example, a value of 0). By setting the pixel value of the pixel of the range corresponding to the region outside the enlarged subject region to the predetermined value, it is possible to suppress the display of a part other than the subject in the tomographic image.

As described above, with the CT apparatus 11 according to the technology of the present disclosure, the margin degree with respect to the subject region is calculated in accordance with the part of the subject to be imaged, and the enlarged subject region to which the peripheral region represented by the margin degree is added to the subject region is calculated. In a case in which the main imaging of the subject is executed, the CT apparatus 11 outputs only the projection data PD of the channel C corresponding to the range of the enlarged subject region in the projection data PD generated by the DAS 25 to the console 17.

Therefore, it is possible to reconstruct the tomographic image including the entire subject by the output projection data PD even in a case in which the output data amount of the projection data PD output to the console 17 is reduced.

One form of the CT apparatus 11 has been described above using the embodiment, but the disclosed form of the CT apparatus 11 is an example, and the form of the CT apparatus 11 is not limited to the range described in the embodiment. Various modifications and improvements can be added to the embodiment without departing from the gist of the present disclosure, and the embodiment to which the modifications or improvements are added is also included in the technical scope of the present disclosure.

For example, the internal processing order in the flowchart of the output processing shown in FIG. 3 and the flowchart of the image display processing shown in FIG. 6 may be changed without departing from the gist of the present disclosure.

In the above-described embodiment, the form has been described in which the output processing and the image display processing are implemented by software processing. However, processing equivalent to the flowchart of each processing may be implemented by hardware. In this case, the processing speed can be increased as compared to a case in which each processing is implemented by software processing.

In the above-described embodiment, the processor 36 is a processor in a broad sense, and includes a general-purpose processor or a dedicated processor (for example, a graphics processing unit (GPU), an application-specific integrated circuit (ASIC), a field programmable gate array (FPGA), a programmable logic device, and the like).

In addition, the operation of the processor 36 in the above-described embodiment may be performed not only by one processor 36 but also by cooperation of a plurality of processors 36 provided at physically separated positions. In addition, the order of the operations of the processor 36 is not limited to only the order described in the above-described embodiment, and may be changed as appropriate.

In the above-described embodiment, the example has been described in which the application program 37 is stored in the storage 33. However, the storage destination of the application program 37 is not limited to the storage 33. The application program 37 can also be provided in a form of being recorded in a computer-readable storage medium.

For example, the application program 37 may be provided in a form of being recorded on an optical disk, such as a compact disk read-only memory (CD-ROM), a digital versatile disk read-only memory (DVD-ROM), and a blue ray disk. In addition, the application program 37 may be provided in a form of being recorded in a portable semiconductor memory such as a universal serial bus (USB) memory and a memory card. The storage 33, the CD-ROM, the DVD-ROM, the blue ray disk, the USB, and the memory card are examples of a non-transitory storage medium.

Further, the processor 36 may download the application program 37 from an external device connected to a network through the communication unit 34 and store the downloaded application program 37 in the storage 33. Further, the present disclosure can also be applied to a program and a program product.

In regard to the above-described embodiment, the following supplementary notes are further disclosed.

### Supplementary Note 1

A CT apparatus comprising: a processor, in which the processor is configured to: set a subject region in an imaging range of a radiation image by using information related to a subject; calculate, before main imaging of the subject, a size of a peripheral region, around the subject region and in a range outside the subject region, in which the subject is estimated to protrude from the subject region due to body movement, as a margin degree with respect to the subject region; and output, during the main imaging of the subject, projection data of a channel corresponding to a range of an enlarged subject region, which is obtained by adding the peripheral region represented by the margin degree to the subject region, to a console.

### Supplementary Note 2

The CT apparatus according to supplementary note 1, in which the processor is configured to calculate the margin degree in accordance with a part of the subject to be imaged.

### Supplementary Note 3

The CT apparatus according to supplementary note 1, in which the processor is configured to calculate the margin degree in accordance with an attribute of a person who is the subject.

### Supplementary Note 4

The CT apparatus according to supplementary note 3, in which the processor is configured to estimate the attribute of the subject from an optical image obtained by imaging the subject.

### Supplementary Note 5

The CT apparatus according to supplementary note 1, in which the processor is configured to reset a display range of the subject region set in advance, in accordance with an instruction of an operator, before the main imaging of the subject.

### Supplementary Note 6

The CT apparatus according to supplementary note 1, in which the processor is configured to set the subject region based on a tomographic model of the subject generated using pre-generation data used for generating a scanogram image or an optical image obtained by imaging the subject.

### Supplementary Note 7

The CT apparatus according to any one of supplementary notes 1 to 6, in which the processor is configured to calculate the margin degree for each imaging direction of the subject, and output, during the main imaging of the subject, projection data of a channel corresponding to a range of the subject region to which the peripheral region represented by the margin degree is added, to the console.

### Supplementary Note 8

The CT apparatus according to any one of supplementary notes 1 to 7, in which the processor is configured to output projection data including a specific channel to be referred to by the console to reconstruct a tomographic image of the subject, in addition to the channel corresponding to the range of the enlarged subject region, to the console.

### Supplementary Note 9

The CT apparatus according to any one of supplementary notes 1 to 8, in which the processor is configured to control an irradiation field limiter that limits a radiation irradiation field such that an outer side of the range of the enlarged subject region is not irradiated with radiation.

### Supplementary Note 10

The CT apparatus according to any one of supplementary notes 1 to 9, in which the processor is configured to adjust a display range of a tomographic image of the subject, which is reconstructed from the projection data, in accordance with the number of channels included in the projection data received by the console, and display the tomographic image of the subject of which the display range is adjusted, on a display device.

### Supplementary Note 11

The CT apparatus according to supplementary note 10, in which the processor is configured to readjust the display range adjusted in accordance with the number of channels included in the projection data, in accordance with an instruction from a user.

### Supplementary Note 12

The CT apparatus according to supplementary note 10 or 11, in which the processor is configured to set a pixel value of a pixel in a range corresponding to a region outside the enlarged subject region in the tomographic image of the subject, to a predetermined value.

### Supplementary Note 13

An operation program of a CT apparatus, the operation program causing a computer to execute a process comprising: setting a subject region in an imaging range of a radiation image by using information related to a subject; calculating, before main imaging of the subject, a size of a peripheral region, around the subject region and in a range outside the subject region, in which the subject is estimated to protrude from the subject region due to body movement, as a margin degree with respect to the subject region; and outputting, during the main imaging of the subject, projection data of a channel corresponding to a range of an enlarged subject region, which is obtained by adding the peripheral region represented by the margin degree to the subject region, to a console.

### Supplementary Note 14

A computer program product including an operation program of a CT apparatus, the operation program causing a computer to: set a subject region in an imaging range of a radiation image by using information related to a subject; calculate, before main imaging of the subject, a size of a peripheral region, around the subject region and in a range outside the subject region, in which the subject is estimated to protrude from the subject region due to body movement, as a margin degree with respect to the subject region; and output, during the main imaging of the subject, projection data of a channel corresponding to a range of an enlarged subject region, which is obtained by adding the peripheral region represented by the margin degree to the subject region, to a console.

### Supplementary Note 15

An operation method of a CT apparatus, the operation method being executed by a computer, the operation method comprising: setting a subject region in an imaging range of a radiation image by using information related to a subject; calculating, before main imaging of the subject, a size of a peripheral region, around the subject region and in a range outside the subject region, in which the subject is estimated to protrude from the subject region due to body movement, as a margin degree with respect to the subject region; and outputting, during the main imaging of the subject, projection data of a channel corresponding to a range of an enlarged subject region, which is obtained by adding the peripheral region represented by the margin degree to the subject region, to a console.

What is claimed is:

1. A CT apparatus comprising:
a processor,
wherein the processor is configured to:
set a subject region in an imaging range of a radiation image by using information related to a subject;
calculate, before main imaging of the subject, a size of a peripheral region, around the subject region and in a range outside the subject region, in which the subject is estimated to protrude from the subject region due to body movement, as a margin degree with respect to the subject region; and
output, during the main imaging of the subject, projection data of a channel corresponding to a range of an enlarged subject region, which is obtained by adding the peripheral region represented by the margin degree to the subject region, to a console.

2. The CT apparatus according to claim 1,
wherein the processor is configured to calculate the margin degree in accordance with a part of the subject to be imaged.

3. The CT apparatus according to claim 1,
wherein the processor is configured to calculate the margin degree in accordance with an attribute of a person who is the subject.

4. The CT apparatus according to claim 3,
wherein the processor is configured to estimate the attribute of the subject from an optical image obtained by imaging the subject.

5. The CT apparatus according to claim 1,
wherein the processor is configured to reset a display range of the subject region set in advance, in accordance with an instruction of an operator, before the main imaging of the subject.

6. The CT apparatus according to claim 1,
wherein the processor is configured to set the subject region based on a tomographic model of the subject generated using pre-generation data used for generating a scanogram image or an optical image obtained by imaging the subject.

7. The CT apparatus according to claim 1,
wherein the processor is configured to calculate the margin degree for each imaging direction of the subject, and output, during the main imaging of the subject, projection data of a channel corresponding to a range of the subject region to which the peripheral region represented by the margin degree is added, to the console.

8. The CT apparatus according to claim 1,
wherein the processor is configured to output projection data including a specific channel to be referred to by the console to reconstruct a tomographic image of the subject, in addition to the channel corresponding to the range of the enlarged subject region, to the console.

9. The CT apparatus according to claim 1,
wherein the processor is configured to control an irradiation field limiter that limits a radiation irradiation field such that an outer side of the range of the enlarged subject region is not irradiated with radiation.

10. The CT apparatus according to claim 1,
wherein the processor is configured to adjust a display range of a tomographic image of the subject, which is reconstructed from the projection data, in accordance with the number of channels included in the projection data received by the console, and display the tomographic image of the subject of which the display range is adjusted, on a display device.

11. The CT apparatus according to claim 10,
wherein the processor is configured to readjust the display range adjusted in accordance with the number of channels included in the projection data, in accordance with an instruction from an operator.

12. The CT apparatus according to claim 10,
wherein the processor is configured to set a pixel value of a pixel in a range corresponding to a region outside the enlarged subject region in the tomographic image of the subject, to a predetermined value.

13. A non-transitory computer-readable storage medium storing an operation program of a CT apparatus, the operation program executable by a computer to execute a process comprising:
setting a subject region in an imaging range of a radiation image by using information related to a subject;
calculating, before main imaging of the subject, a size of a peripheral region, around the subject region and in a range outside the subject region, in which the subject is estimated to protrude from the subject region due to body movement, as a margin degree with respect to the subject region; and
outputting, during the main imaging of the subject, projection data of a channel corresponding to a range of an enlarged subject region, which is obtained by adding the peripheral region represented by the margin degree to the subject region, to a console.

14. An operation method of a CT apparatus, the operation method being executed by a computer, the operation method comprising:
setting a subject region in an imaging range of a radiation image by using information related to a subject;
calculating, before main imaging of the subject, a size of a peripheral region, around the subject region and in a range outside the subject region, in which the subject is estimated to protrude from the subject region due to body movement, as a margin degree with respect to the subject region; and
outputting, during the main imaging of the subject, projection data of a channel corresponding to a range of an enlarged subject region, which is obtained by adding the peripheral region represented by the margin degree to the subject region, to a console.
